(19) United States
(12) Patent Application Publication
(10) Pub. No.: US 2014/0072657 A1
(43) Pub. Date: Mar. 13, 2014

(54) NOVEL PHARMACEUTICAL FORMULATION

(71) Applicant: Helmut Vockner, Kitzbuhel (AT)

(72) Inventor: Helmut Vockner, Kitzbuhel (AT)

(21) Appl. No.: 14/116,104

(22) PCT Filed: May 11, 2012
(86) PCT No.: PCT/EP2012/058738

§ 371 Date: Nov. 6, 2013

(30) Foreign Application Priority Data
May 12, 2011 (EP) 11165857.1

(51) Int. Cl. A61K 31/722; A61K 31/353; A61K 45/06; A61K 31/593

(52) U.S. Cl. CPC A61K 31/722; A61K 31/593; A61K 31/353; A61K 45/06
USPC 424/682; 514/55

(57) ABSTRACT

The present invention provides novel compositions comprising chitosan and vitamin D or chlorhexidin and vitamin D for the treatment of mucosal and epithelial wounds and for the treatment or prevention of oral, mucosal or dermal infections or inflammations.

The present invention provides novel compositions comprising chitosan and vitamin D or chlorhexidine and vitamin D for the treatment of mucosal and epithelial wounds and for the treatment or prevention of oral infections and other cutaneous or mucosal inflammations.

### Cutis

Cutis is the combined term for the epidermis and the dermis, the two outer layers of the skin. Underneath is the subcutis. The epidermis is the outer layer of the skin, composed of terminally differentiated stratified squamous epithelium, acting as the body's major barrier against an inhospitable environment. The epidermis is avascular, nourished by diffusion from the dermis, keratinocytes are the major constituent. The stratified squamous epithelium is maintained by cell division within the basal layer. Differentiating cells slowly displace outwards through the stratum spinosum to the stratum corneum, where anucleate corneal cells are continually shed from the surface (desquamation). The dermis is a layer of skin between the epidermis and subcutaneous tissues, and is composed of two layers, the papillary and reticular dermis. Structural components of the dermis are collagen, elastic fibers, and extracellular matrix.

### Mucosa and Endothelium:

The mucosal membranes are linings of mostly endodermal origin, covered in epithelium, which are involved in absorption and secretion. They line various body cavities that are exposed to the external environment and internal organs. Epithelium is a tissue composed of cells that line the cavities and surfaces of structures throughout the body. It lies on top of connective tissue, and the two layers are separated by a basement membrane. Epithelium is often defined by the expression of the adhesion molecule e-cadherin. Loss of function of e-cadherins contributes to progression in cancer by increasing proliferation, invasion, and/or metastasis. The blood-brain barrier in the central nervous system CNS built of endothelial cells restrict the diffusion of microscopic objects (e.g. bacteria) and large or hydrophilic molecules into the cerebrospinal fluid CSF, while allowing the diffusion of small hydrophobic molecules (O₂, hormones, CO₂). Cells of the barrier actively transport metabolic products such as glucose across the barrier with specific proteins.

Endothelial cells forming capillaries in the CNS differ from those in the rest of the body in two respects. First, they are able to form tight junctions, restricting paracellular flux, and, second, they have very few endocytotic vesicles, limiting transcellular solute movement from the blood to the brain interstices (Rubin, R. R, Staddon, J. M. Annu Rev Neurosci. 1999; 22:11-28. and Staddon J M, Rubin L L., Curr Opin Neurobiol. 1996 October; 6(5):622-7). As a result, the blood-brain barrier impedes entry of virtually all blood molecules, except those that are small and lipophilic, such as steroids. A dense basement membrane and astrocyte processes, termed end-feet, surround capillary endothelial cells, further contributing to the blood-brain barrier.

Periodontium is the supporting structure of a tooth, helping to attach the tooth to surrounding tissues and to allow sensations of touch and pressure. It consists of the cementum, periodontal ligaments, alveolar bone, and gingiva. Periodontal ligaments connect the alveolar bone to the cementum of the tooth. Alveolar bone surrounds the roots of teeth to provide support and creates what is commonly called an alveolus, or “socket”. Lying over the bone is the gingiva or gum, which is readily visible in the mouth. Cell Adhesion Molecules (CAMs) are proteins located on the cell surface involved with the binding with other cells or with the extracellular matrix (ECM) in the process called cell adhesion. These proteins are typically transmembrane receptors and are composed of three domains: an intracellular domain that interacts with the cytoskeleton, a trans-membrane domain, and an extracellular domain that interacts either with other CAMs of the same kind (homophilic binding) or with other CAMs or the extracellular matrix (heterophilic binding). The major types of molecular processes that control cellular differentiation involve cell signalling. Dedifferentiation is a natural part of the immune response, wound healing and tissue repair.

Cell signalling is part of a complex system of communication that governs basic cellular activities and coordinates cell actions. The ability of cells to perceive and correctly respond to their microenvironment is the basis of development, tissue repair, and immunity as well as normal tissue homeostasis. Complex multi-component signal transduction pathways provide opportunities for feedback, signal amplification, and interactions inside one cell between multiple signals and signalling pathways. Cellular signalling pathways like the mitogen-activated protein kinase (MAPK) pathway couple intracellular responses to the binding of growth factors to cell surface receptors and CAMs. This pathway is very complex and includes many components.

Mitogen-activated protein (MAP) kinases are serine/threonine-specific protein kinases that respond to extracellular stimuli (mitogens, osmotic stress, heat shock and proinflammatory cytokines) and regulate various cellular activities, such as gene expression, mitosis, differentiation, proliferation, adaptation to drugs and cell survival/apoptosis.

The classical ERK MAPK pathway is responsible for cell proliferation and differentiation whereas the JNK and p38 pathways lead to inflammation, dedifferentiation, disjunction and apoptosis (programmed cell death). In many cell types, activation of these pathways promotes cell division. PKC is used by many receptors to regulate the MAP kinase pathway, alone or with other mechanisms and may act at several steps in the cascade. Other sites of action of PKC are likely to be either farther upstream or at the level of MAP kinase inactivation.

In cell biology, microtubule-associated proteins (MAPs) are proteins that interact with the microtubules of the cellular cytoskeleton. They are both stabilizing and destabilizing microtubules, guiding microtubules towards specific cellular locations, cross-linking microtubules and mediating the interactions of microtubules with other proteins in the cell.

Keratin refers to a family of fibrous structural proteins. Keratin is an intermediate filament; when assembled in bundles, it is tough and insoluble forming hard, unmineralized structures. Like actin filaments in microtubules, intermediate filaments function in the maintenance of cell-shape by bearing tension. Microtubules resist compression. It may be useful to think of micro- and intermediate filaments as cables, and of microtubules as cellular support beams. Intermediate filaments organize the internal tridimensional structure of the cell, anchoring organelles and serve as structural components in some cell-cell and cell-matrix junctions.

Wound healing, or wound repair, is an intricate process in which the skin or another organ repairs itself after injury. In normal skin and mucosa, the epithelium, the outermost layer, and the underlying connective tissue exist in steady-state equilibrium, forming a protective barrier against the external environment, the key function of innate immunity. Tight Junctions have an organizing role in epithelial and mucosal polarization and establish an apico-lateral barrier to the diffusion of solutes through the intra- and paracellular space (gate function). They also restrict the movement of lipids and membrane proteins between the apical and the basolateral membrane (fence function).

Once the protective barrier is broken, the normal physiologic process of wound healing is immediately set in motion. The cellular reaction after injury depends on the tissue type as well as the extent of the wound and level of infection or inflammation. Ultimately these processes are resolved or dampened leading to a mature wound and macroscopic scar formation. Although inflammation and repair mostly occur along a proscribed course, the sensitivity of the process is underscored by the consequences of disruption of the balance of regulatory cytokines. Consequently, cytokines, which are central to this constellation of events, have become targets for therapeutic intervention to modulate the wound healing process. Depending on the cytokine and its role, it may be appropriate to either enhance (recombinant cytokine, gene transfer) or inhibit (cytokine or receptor antibodies, soluble receptors, signal transduction inhibitors, antisense) the cytokines to achieve the desired outcome.

It is also possible to influence cytokine profile and cell receptor profile, junctional complexes and differentiation by standard pharmaceuticals, as shown below.

Healing of both oral mucosal and dermal wounds proceeds through the same stages, including hemostasis, inflammation, proliferation, reepithelialization, angiogenesis and remodeling of the collagen matrix. Each stage of the wound healing response, i.e. hemostasis, inflammation, repair, angiogenesis, remodeling, is controlled by the onset of several genes, molecular pathways and distinct cytokine profiles, leading to different development stages of adhesive junctional complexes, cell cycle and tissue differentiation.

Impaired wound healing through reduced vascularisation and tissue differentiation is found e.g. in chronic venous ulcerous wounds of diabetics, epidermolysis bullosa EB and biofilm infection of periodontal tissues:

Impaired healing results from interruption of the healing process at explicit stages by bacterial infections or poor nutrition of the affected tissue, leading to disjunction, reduced expression of adhesive complexes and extracellular matrix components in addition to the constant degradation of matrix proteins and growth factors in the wound exudates.

Different cytokine profiles lead to expression of different, i.e. chronical wound forms:

In (diabetic) venous ulcers, different to regular wound repair, Vascular Endothelial Growth Factor (VEGF) induced Nitric Oxide (NO) is reduced leading to lower neovascularisation and atrophic state of the wound with low proliferation of cutaneous structures; in periodontal disease NO induces vascularisation resulting in swelling, gum bleeding and dedifferentiation and degradation of the mucosal barrier and supportive structures.

Mechanisms of Wound Healing in Healthy People Versus People with Diabetes:

In healthy individuals, the acute wound healing process is guided and maintained through integration of multiple signals released by keratinocytes, fibroblasts, endothelial cells, macrophages, and platelets and other cells. During wound-induced hypoxia, VEGF released by macrophages, fibroblasts, and epithelial cells induces the activation of NOS in the bone marrow, resulting in an increase in NO levels, which triggers the mobilization of bone marrow Endothelial Progenitor Cells to the circulation. Released chemicals (SDF-1α) promote the homing of these EPCs to the site of injury, where they participate in neovasculogenesis. Gallagher and colleagues show that NOS activation in the bone marrow is impaired, which directly limits EPC mobilization from the bone marrow into the circulation. They also show that SDF-1α expression is decreased in the diabetic wound, which prevents EPC recruitment to wounds and therefore limits wound healing (Gallagher, K., J Clin Invest. 2007 May 1; 117(5): 1249-1259).

### Epidermolysis Bullosa:

Epidermolysis bullosa (EB) is a rare genetic disorder caused by a mutation in the keratin genes. The disorder is characterized by the expression of defective keratins in the basal cell layer of the epidermis and presence of extremely fragile skin and recurrent blister formation, resulting from minor mechanical friction or trauma. In normal individuals, keratin filaments impart mechanical strength to epithelial tissues in part by anchoring the intermediate filaments at sites of cell-cell contact, called desmosomes, or cell-matrix contact, called hemidesmosomes. Basement membranes are sheet-like depositions of Extra Cellular Matrix ECM on which various epithelial cells rest.

In people born with EB, the epidermis or two skin layers or junctional complexes to the basement membrane lack the anchors that hold them together, and any action that creates friction between the layers (like rubbing or pressure) will create blisters and painful sores. Sufferers of EB have compared the scores to second- to third-degree burns. Keratin chains are coexpressed during differentiation of simple and stratified epithelial tissues. Clumping of the tonofilaments occurs in association with early blister formation. The severity of the clinical disease appears to correlate with the degree of disruption of keratin filament or junctional complex formation. These mutations result in poor keratin filament formation or not fully differentiated junctional complexes, thus contributing to cell and tissue fragility. Open wounds on the skin heal slowly or not at all, often scarring extensively, and are particularly susceptible to infection.

Gingivitis and Periodontitis refer to a number of inflammatory diseases affecting the tissues that surround and support the teeth and are caused by microorganisms that adhere to and grow on the tooth's surfaces, form biofilm colonies out of which anaerobic species enter the periodontal tissues resulting in partly destructive host immune reactions.

In Gingivitis inflammation along the gumlines leads to swollen and redish mucosa reaction with exsudation and bleeding. A combination of suboptimal doses of chemically modified non-antimicrobial tetracyclines and bisphosphonates were shown to inhibit endotoxin-induced gingival collagenase in rats (Llavaneras A. et al., J. Periodontol., 2001, 72, 8, pp. 1069-1077).

Periodontal infection leads to disjunction of the supportive periodontal ligament, pocket formation and attachment and alveolar bone loss.

Periodontitis is very common, is widely regarded as the second most common disease worldwide, after dental decay, and has prevalence in Europe and the United States of 30-50% of the population aged above 35 years, of which about 15%-20% suffer from severe forms. Various concepts in periodontal therapy have been tried, specifically antimicrobial therapies using compositions based on single active compounds like chlorhexidine, phenolic agents or antiseptics or antimicrobial agents or NSAIDs (Greenwell H. and Bissada, N., Drug, 2002, 62(18) pp. 2681-2687; Norowski P. and Baumgardner J., J. Biomed. Mat. Res., Past B: Applied Biomaterials, 88, 2, 2009, pp. 530-543, Badran Z. et al., Oral Health & Preventive Dentistry, 2009, 7, 1, pp. 3-12).

Periimplantitis is caused by Biofilm ingrowth on the implant surface, disintegration of the tissue interface, infection of the surrounding tissue and implant loss.

As indicator of periodontal status pocket depth is used according to Treatment Need codes 3 and 4 in Community Periodontal Index—Treatment Needs (CPITN). This code is applied to every treated tooth or implant.

Periodontal pockets: shallow (Code 3: 4-5 mrn) and deep (Code 4: 6 mm or more).

TABLE 1

| CPITN | | |
|---|---|---|
| Codes | Periodontal Condition | Treatment Need: 0-III |
| 0 | No bleeding | No treatment |
| | No calculus | |
| | No pathological pocket | |
| 1 | Bleeding on probing gingival margin | I Motivation: biofilm |
| | No calculus | control |
| | No pathological pocket | |
| 2 | Presence of calculus (sub- or | II Motivation + |
| | supragingival) with or without bleeding | Scaling polishing: |
| | No pathological pocket | Biofilm control |
| 3 | Pathological pocket of 4-5 mm with or | II complex treatment |
| | without bleeding and calculus | |
| 4 | Pathological pocket of 6 mm or more | III complex treatment |
| | with or without bleeding and calculus | |

Chinese patent application CN-97103254A describes an ointment prepared from Hibitane and cod liver oil for topical administration.

JP2007 084471A describes a chlorhexidine containing solution for oral administration.

The effect of vitamin D3 on dermal wound healing is described by Ramesh K. V. et al. (Indian J. of Exp. Biology, 1993, 31, 778-779).

WO2008/073174 discloses the treatment of dermal diseases using a combination of an antimicrobial peptide and vitamin D3. Transdermal compositions comprising tertiary amides are described in US2004/122105A1. Amongst different topical gels, a chlorhexidine-containing gel for treatment of periodontitis is described by Cohen R. E. et al. (Clinical Preventive Dentistry, 1991, 13, 5, 20-24.

JP2004210675 discloses a composition comprising, amongst other agents, vitamin D3 and its use for the treatment of osteoporosis.

Standard therapy for periodontitis as presently recommended for Class 3+4 comprises complex treatment consisting of motivation, biofilm control by patient (e.g. CHX 1 wt % brushing for 2 weeks) and in office polishing, ultrasonic scaling of tooth surface and pocket, administration of antibiotics, splinting of mobile teeth, and surgical procedures (bone augmentation, guided bone regeneration, tissue graft, implant therapy). Although this method has proved to be fairly successful in treating individuals, there is still a high recurrence rate.

There is a constant need for an efficient treatment that can support wound healing of the epidermis or the mucosa, specifically to have a composition that can re-establish the healthy tissue condition around the affected tooth and can, as a result, render dental surgery or tissue graft unnecessary.

There is no causative treatment for EB; symptomatic wound care helps to cope with the difficulties for the “butterfly disease” affected.

It is an object of the invention to provide a novel composition that can fulfil the unmet needs. It is a further object of the invention to provide a method for treating epithelial wounds or oral infections or inflammations using such compositions.

### SUMMARY OF THE INVENTION

The object is solved by the subject matter as claimed. According to the invention there is provided a topical composition comprising vitamin D and chitosan or chlorhexidine. Specifically, the vitamin D is vitamin D3.

These active ingredients may be combined into a gel, cream, solution, rinse, gauge or other pharmaceutically acceptable carrier which can be applied topically to or onto the wounded sides of inflammation or infection.

Although the single components as used for the inventive composition are known in the field of anti-inflammatory and antimicrobial as well as regenerative treatments, it was surprisingly shown by the inventor that the present compositions show a highly therapeutic effect especially in the treatment and prevention of epithelial wounds, oral infections or inflammations and show tissue regenerative capacity.

The invention is also directed to a method for treating epithelial or mucosal infections or inflammations employing the above described topical preparations.

Systemic application of each composition could provide help to treat inflammation in body compartments difficult to reach as the CNS being protected by a dense blood-brain barrier consisting of junctional complexes.

Because NO production via inducible NO synthase in articular chondrocytes plays a central role in the pathophysiology of arthritis by causing inflammation, apoptosis, dedifferentiation, and the activation of matrix metalloproteinases, the inventor's results suggest that OPCs comparable to NSAIDs have protective effects on cartilage damage, not only by alleviating inflammation but also by inhibiting NO-induced apoptosis and dedifferentiation of articular chondrocytes (Yoon, K. 2003, The Journal of Biological Chemistry, 278, 15319-15325) and immunomodulatory agents restore the chronic inflammatory response. Barrier function is restored as well as normal tissue homeostasis and even regeneration when combining these agents.

The term “nitrosative stress” is put aside the oxidative stress within the last years (Hausladen, Privalle et al. 1996, Nat. Struct. Biol., 5, 247-249, Stamler & Hausladen 1998, Cell, 86, 719-729). It was shown that many expressions of cell pathology which were until now explained as reactions to oxidative stress could be at least partly explained as a result of increased production of Nitric Oxide NO.

### The Dualistic Nature of the Immune System:

### “Jin-Yang” Cross-Regulation—Balance of Th1/Th2 Immunity

IL2-IFN-γ-TNFβ-IL-12-TH1-cellular cytotoxic, (peripheral IGG)
   TH0 [symbol]
IL-6-IL-5-IL-10-Il-4-TGFβ1-TH2: humoral (mucosal IGA)

Micro environmental factors stimulate T-cells to express either T-helper 1 (Th1) or T-helper 2 (Th2) cytokines which are associated with “cellular” and “humoral” immunity, respectively. Other alignments of cytokines may contribute to cross-regulation of immune responses associated with parenteral or mucosal immunization. The cytokines IL-12 and TGF beta 1 are predominant influences in “peripheral” and “mucosal” lymphatic tissues. Thus expression of these cytokines affect T cells and B cells in such a way that proliferating B cells become committed to secrete “peripheral” IgG or “mucosal” IgA, respectively.

The mucosal immune system is a complex and redundant system that generates large amounts S-IgA as well as cell-mediated immunity at mucosal surfaces to prevent pathogen infiltration and inflammation. Limited Cytotoxic T-Lymphocyte CTL activity at mucosal surfaces is a built-in mechanism to protect the mucosal epithelium from damage. Anti-genic exposure at mucosal sites activates mucosal B and T-lymphocytes to emigrate from the inductive site and home to various mucosal effector sites. Antigen-specific CTL responses at mucosal surfaces are dictated by induction of CTL locally. CTLs in immunologically privileged sites fail to differentiate into fully functional CTL, unless exposed to antigen (Ksander B R, 1990, J Immunol. 1990 Oct. 1; 145(7):2057-63).

Regulatory T-cells (T_{reg}) regulate both acquired and innate immunity through multiple modes of suppression. Immunomodulation by CD25_CD4_T cells in which T_{reg} activity is contextual along a continuum of dendritic cell DC maturation and TLR-induced activation, and mechanisms contributing to the reversal of T_{reg} suppression and anergy are separable, and independently modulated by proinflammatory cytokines produced by DCs (Kubo Takekazu 2004, The Journal of Immunology, 2004, 173: 7249-7258).

Selective targeting of T_{reg}-cell trafficking and compartmentalization is therapeutically beneficial:

### Vitamin D Biology

Vitamin D and its metabolites are transported in the circulation by a specific binding protein, vitamin D binding protein (DBP), which is normally present in large excess. Active vitamin D, 1α,25-dihydroxyvitamin D3 (1,25(OH)₂D₃ or calcitriol), is generated by hepatic 25-hydroxylation and renal 1α-hydroxylation of inactive precursors. Calcitriol alters gene expression by binding with high affinity to its intracellular receptor, the vitamin D receptor (VDR), which acts as a nuclear transcription factor. Binding to the VDREs (vitamin D response elements) may promote transcription, as is the case with osteocalcin in the osteoblast, or inhibit transcription, as for parathyroid hormone (PTH) in the parathyroids, by either enhancing or repressing the activity of transcription machinery. Additionally, 1,25(OH)₂D₃ appears to bind to one or more cell surface receptors that, through second messenger pathways, mediate certain non-genomic effects. While the principal role of 1,25(OH)₂D₃ in mineral homeostasis is effected by its influence on ‘classic’ targets, namely gut, bone and parathyroid glands, its actions extend much further. This has helped to re-ignite interest in novel therapeutic applications and in vitamin D biology as a whole. (Chapuy M C, Osteoporos. Int. 2002 March; 13(3):257-64.)

Vitamin D insufficiency and low calcium intake contribute to increase parathyroid function and bone fragility in elderly people. Calcium and vitamin D supplements can reverse secondary hyperparathyroidism thus preventing hip fractures, as proved by Decalyos I. Decalyos II is a 2-year, multicenter, randomized, double-masked, placebo-controlled confirmatory study. The intention-to-treat population consisted of 583 ambulatory institutionalized women (mean age 85.2 years, SD=7.1) randomized to the calcium-vitamin D3 fixed combination group (n=199); the calcium plus vitamin D3 separate combination group (n=190) and the placebo group (n=194). Fixed and separate combination groups received the same daily amount of calcium (1200 mg) and vitamin D3 (800 IU), which had similar pharmacodynamic effects. Both types of calcium-vitamin D3 regimens increased serum 25-hydroxyvitamin D and decreased serum intact parathyroid hormone to a similar extent, with levels returning within the normal range after 6 months. In a subgroup of 114 patients, femoral neck bone mineral density (BMD) decreased in the placebo group (mean=−2.36% per year, SD=4.92), while remaining unchanged in women treated with calcium-vitamin D3 (mean=0.29% per year, SD=8.63). The difference between the two groups was 2.65% (95% CI=−0.44, 5.75%) with a trend in favor of the active treatment group. No significant difference between groups was found for changes in distal radius BMD and quantitative ultrasonic parameters at the os calcis. The relative risk (RR) of HF in the placebo group compared with the active treatment group was 1.69 (95% CI=0.96, 3.0), which is similar to that found in Decalyos I (RR=1.7; 95% CI=1.0, 2.8). Thus, these data are in agreement with those of Decalyos I and indicate that calcium and vitamin D3 in combination reverse senile secondary hyperparathyroidism and reduce both hip bone loss and the risk of hip fracture in elderly institutionalized women (Pepper C, Blood. 2003 Apr. 1; 101(7):2454-60. Epub 2002 Nov. 21.)

Immature dendritic cells are stimulated by Vit D3 to differentiate into tolerogenic dendritic cells (Penna G, J Immunol 2000; 164:2405-11) and to induce regulatory T-cells (Penna G, 2007) to inhibit inflammatory T-cells (Barrat F J, J Exp Med 2002; 195: 603-16). Vit D3 inhibits immature dendritic cell differentiation into mature dendritic cells (Griffin 2004) and expansion of inflammatory T-cells (Griffin M D J Steroid Biochem Mol Biol 2004; 89-90: 443-8) which stimulate inflammation and are inhibited to promote inflammation by D3 as well (Xing N, Biochem Biophys Res Commun. 2002; 297: 645-52).

Prolonged induction of excessive levels of inflammatory mediators and deregulated recruitment of leukocytes resulting in tissue damage contribute to the pathogenesis of chronic disease states, such as periodontitis. A central proinflammatory pathway is initiated by microbial stimulation of toll-like receptors (TLRs) on innate immune cells leading to activation of the nuclear factor-kappa B (NF-ₖB) (Ulevitch R J, Nat Rev Immunol 2004; 4:512-520). NF-ₖB regulates the expression of genes encoding inflammatory cytokines, chemokines, and adhesion molecules (Medzhitov R, N Engl J Med 2000; 343:338-344). The TLR/NF-ₖB pathway plays a major inductive role in the inflammatory response to periodontal pathogens (Hajishengallis G, Immunol Invest 2004; 33:157-172; Gibson F C 3rd, Circulation 2004; 109:2801-2806; Muthukuru M, Infect Immun 2005; 73:687-694; Bainbridge B W, Acta OdontolScand 2001; 59:131-138) and thus constitutes a reasonable target to control periodontal inflammatory activity.

Various plants have been shown to have antiinflammatory properties, and the active ingredients have been isolated (Calixto J B, Planta Med 2003; 69:973-983). The curry spice curcumin from the plant Curcuma longa displays protective anti-inflammatory action in animal models of arteriosclerosis and Alzheimer's disease. Plant-derived anti-inflammatory compounds present a relatively inexpensive and safe alternative to synthetic drugs (Calixto J B, Planta Med 2003; 69:973-983). Black Elderberry, Chokeberry, Black Raspberries, Blueberries, European Blackcurrant contain high amounts of flavonoids and oligomeric polycyclic cyanids (OPC) found also in cranberries, cherries, wine seed, grapes, pine bark (quercus), green tea, Propolis etc.

Black Elderberry (Sambuccus nigra) aequous extract (SNAE) displays potent anti-inflammatory activities in professional phagocytes activated by periodontogenic bacteria (Porphyromonas gingivalis, Aggregatobacter actinomycetemcomitans LPS and fimbriae), including inhibition of 1) proinflammatory cytokine release, 2) integrin activation, and 3) oxidative burst. S. nigra contains quercetin, anthocyanins and rutin. SNAE can inhibit IL-1 and TNF (Harokopakis E, J Periodontol 2006; 2:271-279).

Propolis is a composition of different plant exsudates collected by bees mixed with and chemically altered by beeswax and E-glucosidase they secrete during collection. More specifically, propolis constituents include about 10% essential oils, 5% pollen, and 15% various organic polyphenolic compounds including flavonoids and phenolic acids (Greenaway W, A Report of Work at Oxford 1990; 117-118; Xing X, The Food Industry 2008; 01). The polyphenolic content of propolis is considered to contribute more to the observed healing effects than other propolis constituents.

Oligomeric polycyclic cyanids (OPC cyanidin bioflavonoids) inhibit platelet function (Murphy, K. 2003, Pütter, M. 1999) and, selectively bind to collagen and elastin and aid in the production of endothelial NO (Nishioka, K. 2007).

Their effects on cytokine regulation are less well studied, although recent data suggest that they may play a role in modulating cytokines involved in acute inflammatory responses (Rotondo et al. 1998, Sanbongi et al. 1997, Sato et al. 1997).

Studies have so far identified a reduction in intracellular reactive oxygen species, which activate nuclear transcription factor-KB, and an inhibition of cytoplasmic calcium ions in response to these polyphenols (Rotondo et al. 1998, Sato et al. 1997).

### DETAILED DESCRIPTION OF THE INVENTION

A composition is provided that comprises chitosan and vitamin D.

As a further embodiment, a composition comprising chlorhexidine and vitamin D is provided.

According to specific embodiments, compositions comprising vitamin D and chitosan may additionally comprise chlorhexidine and compositions comprising vitamin D and chlorhexidine may additionally comprise chitosan as further active agent.

Specifically, the vitamin D as used in the composition is vitamin D3. Said compositions may optionally further comprise oligomeric proanthocyanide (OPC).

The inventive compositions are free of NSAID and bisphosphonate.

Specifically, the inventive compositions are formulated as a pharmaceutical composition or incorporated in a device.

Chitosan is a linear polysaccharide of β(1-4) linked D-glucosamine and N-acetyl-D-glucosamine and was shown by the inventor to be a highly efficient gelling agent and exhibits highly effective anti-microbial and regenerative characteristics.

Chitosan may be present in the present composition in the amount of about 0.001 to 50% by weight, preferably in the amount of 0.01 to 20%, more preferred in the amount of about 0.1 to 10%, even more preferred in the amount of about 1% by weight.

Specifically, chitosan is present in the form of soluble chitosan ascorbate.

According to a specific embodiment of the invention, chlorhexidine can be present in the inventive composition in the range of 0.01 to 10 wt %, preferably in the range of 0.01 to 5 wt %, preferably in the range of 0.1 to 2 wt %, more preferred in an amount of about 1%.

Analogues or derivatives of chitosan, chlorhexidine, vitamin D, specifically vitamin D3, or OPC are contained within the scope of the invention. The term “analogues” includes also derivatives and analogues of said substances. The terms “analogue” or “derivative” relate to a chemical molecule that is similar to another chemical substance in structure and function, often differing structurally by a single element or group, which may differ by modification of more than one group (e.g. 2, 3, or 4 groups) if it retains the same function as the parental chemical. Such modifications are routine to skilled persons and include, for example, additional or substituted chemical moieties, such as esters or amides of an acid, protecting groups such as a benzyl group for an alcohol or thiol, and tert-butoxylcarbonyl groups for an amine. Derivatives can also include conjugates, such as biotin or avidin moieties, enzymes such as horseradish peroxidase and the like, and radio-labeled, bioluminescent, chemoluminescent, or fluorescent moieties. Further, moieties can be added to the agents described herein to alter their pharmacokinetic properties, such as to increase half-life in vivo or ex vivo, or to increase their cell penetration properties, among other desirable properties. Also included are prodrugs, which are known to enhance numerous desirable qualities of pharmaceuticals (e.g. solubility, bioavailability, manufacturing, etc.).

The term “derivative” also includes within its scope alterations that have been made to a parent sequence including additions, deletions, and/or substitutions that provide for functionally equivalent or functionally improved molecules.

Vitamin D3 which, when binding to vitamin D receptor, has been shown to increase the activity of natural killer cells, enhance the phagocytotic activity of macrophages, inhibit inflammatory cytokine production, reduce the inflammatory response of TH1-cells, increase antimicrobial peptides (e.g. cathelicidin (LL-37), and has positive effects on bone formation and preservation.

Vitamin D, specifically vitamin D3, may be present in the inventive composition, but is not limited to, in the form of cholecalciferol (Calciol), calcifediol (calcidiol, 25-hydroxycholecalciferol, or 25-hydroxyvitamin D), 22-oxacalcitriol Maxacalcitol (OCT), Paricalcitol (vitamin D2 derived sterol lacking the carbon-19 methylene group), Doxercalciferol (1α-hydroxyvitamin D2), alfacalcidol (1α-hydroxyvitamin D3), Dihydrotachysterol₂ (DHT₂), tacalcitol, ergocalciferol (D2), calcidiol, calcipotriol MC 903, ergosterol, lumisterol, sitocalciferol, alfacalcidiol, inecalcitol, EB 1089 (Seocalcitol), ED 71, Gemini D3 analog (Gemini is a 1α,25-dihydroxyvitamin D³ analog with two identical side chains that, despite its significantly increased volume, binds to the VDR and can function as a potent agonist), or calcitriol (1,25-dihydroxycholecalciferol or 1,25-dihydroxyvitamin D₃). Cholecalciferol may be the most preferred form.

Specifically, vitamin D may be present in said composition in the range of 0.00001 to 1%, preferably in the range of 0.00005 to 0.05%, preferably in the range of 0.0001 to 0.01% by weight, more preferred in an amount of about 0.0025% by weight of the composition.

Vitamin D3 cofactors may be contained in the composition, like for example vitamin K2 and magnesium.

Specifically, OPC may present in said composition in the form of a standardized cowberry/lingonberry, propolis or elderberry extract in the range of 50 to 0.00001% by weight, preferably in the range of 20 to 0.0001% by weight of the composition, preferably in the range of 10 to 0.001% by weight of the composition preferably about 10 wt %. Alternatively, further active compounds like curcumin, ginger or calcium may also be present in the composition.

Specifically the inventive composition can comprise

Chitosan, specifically in an amount of about 0.1 to 3 wt %
Vitamin D3, specifically in an amount between 0.0001-0.001 wt %

Alternatively the inventive composition can comprise

Chlorhexidine, specifically in an amount between 0.01 and 20 wt %
Vitamin D3, specifically in an amount between 0.0001-0.001 wt %

According to a further alternative, the composition may comprise

Chitosan, specifically in an amount between 0.1 to 3 wt %
Vitamin D3, specifically in an amount between 0.0001-0.001 wt %
OPC, specifically in an amount between 0.0001 and 50 wt %

As a further alternative, the composition may comprise:

Chlorhexidine, specifically in an amount between 0.01 and 20 wt %
Vitamin D3, specifically in an amount between 0.0001-0.001 wt %
OPC, specifically in an amount between 0.0001 and 50 wt %

The inventive composition is preferably free of NSAID and bisphosphonate.

According to a specific embodiment of the invention, the composition contains chitosan and vitamin D, optionally together with OPC as sole active compounds, free of any further active compounds.

According to a further specific embodiment of the invention, the composition contains chlorhexidine and vitamin D, optionally together with OPC as sole active compounds, free of any further active compounds.

The term “active compound” is defined as any compound which is effective to achieve a desired therapeutic or prophylactic result. According to the present invention an active compound effects the treatment of mucosal and epithelial wounds or effects the treatment or prevention of oral infections and other cutaneous or mucosal inflammations. Thus, adjuvants or additives, suspending or scaffolding agents known for production of cosmetic, pharmaceutical or medical compositions are not defined as active compounds.

The specific dose of compounds administered according to this invention to obtain therapeutic or prophylactic effects will, of course, be determined by the particular circumstances surrounding the case, including, for example, the specific route of administration and response of the individual patient, the condition being treated and the severity of the patient's symptoms. In general, the compounds of the invention are most desirably administered at a concentration that will generally afford effective results without causing any serious side effects.

The composition according to the embodiment is a solid or liquid composition. The active ingredients of the composition can be combined into a gel, hydrogel, polymer, cream, solution, rinse or other pharmaceutically acceptable carrier which can be applied topically to the sides of epithelial wounds, mucosal inflammation or infection. Systemic application of parts of the composition is possible.

More specifically a gel, polymer or hydrogel is the most preferred administration form of the inventive composition, specifically if the composition is administered to a periodontal pocket or a wound of an individual.

The addition of suspending or scaffolding agents to the carrier like, for example, xanthan gum, methylcellulose, hydroxymethylcellulose or hydroxypropylcellulose, or covering agents like PEG-hydrogels can be advantageous when compounding the carrier. In some cases, depending on the carrier, the active agents are first solubilised and then incorporated into the carrier.

Scaffolds capable of supporting three-dimensional tissue formation are critical to recapitulating the in vivo milieu and allowing cells to influence their own microenvironments. Scaffolds usually serve to allow cell attachment and migration, deliver and retain cells and biochemical factors, enable diffusion of vital cell nutrients and expressed products and exert certain mechanical and biological influences to modify the behaviour of the cell phase. Examples of these materials are processed cellular and acellular ECM, proteins like collagen, elastin, fibronectin, laminin, glycosaminoglycans (GAGs), fibrin, soybean proteins, natural polymers and polysaccharidic materials, like chitosan, alginate, dextran, cellulose, starch, hyaluronan, silk fibroin, and some polyesters, polylactic acid (PLA), polyglycolic acid (PGA), polycaprolactone (PCL) and polyhydroxyalkanoates.

In addition to mimicking extracellular matrix characteristics, as well as covering, moist keeping characteristics, these bio-functionalized scaffold and covering ingredients can be applied to every wound related site.

The inventive compositions can be used for the treatment of epithelial wounds as well as for the prevention or treatment of infections or inflammations of the mucosa, specifically of oral infections or inflammations, more specifically for the treatment of periodontitis and other related diseases, for example oral mucositis, gingivitis or periodontitis, periimplantitis and postoperative or chronic dermal and mucosal wound healing.

The inventive compositions can be used for the prevention or treatment of any chronic inflammation which may lead to dedifferentiation of the tissues, in case of mucosal and periodontal sites, inflamed tooth extraction sites used for immediate implant placement to a loss of barrier function and to chronic infection of the underlying connective tissue. Swelling, redness, edema and pain with pocket formation along the tooth root or implant surface, attachment loss, bone resorption and delayed wound healing result in tissue destruction, tooth loss and in dermal wound sites in scar formation instead of regular tissue differentiation and regeneration can be treated or prevented as well with the present composition.

Furthermore the inventive compositions can be used for prevention or treatment of any acute or chronic mucosal or dermal atraumatic or traumatic inflammation or infection as well as chronic inflammatory tissue destruction or diabetical or other wound healing disturbances.

The present invention further provides ready to use of devices for delivering the compositions according to the invention into a periodontal pocket, to an implant placement, mucosal or wound surface or inflammatory area.

Said devices are well known in the art specifically for oral and dermal treatments and can be in the form of a syringe, gel, cream or naturally derived or synthetic matrices,

Hydrogels, polymers in any shape or configuration, gauze, sheets or filler material as known in the art can also be useful for administration of the compositions.

By increasing moisture content, hydrogels consisting of cross-linked, wound secretion absorbing bio- or synthetic polymers have the ability to help cleanse and debride necrotic tissue. A moist wound dressing method for example as hydrogel matrix prevents wound contraction. Using this wound dressing, the suitability of e.g. PEG-hydrogels for the application in wound healing was demonstrated. Synthetic materials promote epidermal healing even better than naturally derived fibrin matrices, since the degree of reepithelialization was more than 4-fold increased in PEG hydrogels.

The treatment of the oral infections and inflammations can be performed for example together with treatment procedures stage 1+11 instruction for self administration by the patient of the gelous composition into the diseased pockets (Code 3+4 CPITN) out of a syringe is administered for several weeks (2-8) depending on resulting pocket depth and repeated if necessary.

As periodontal disease develops on anaerobically modified biofilm level, it is necessary to re-establish a healthy microbial community on the tooth surface. High and frequent sugar intake allows excessive plaque development and anaerobic shift at the gumline, facilitated by attachment loss and exposure of dentine bone protein structure allowing anaerobic receptor-adhesin coadhesion of red complex anaerobic pathogens.

As periodontitis is a multifactorial disease resulting from biofilm infection of the periodontal structures, affecting immune defense and tissue differentiation, cytokine signalling and resulting in chronic inflammation and destruction, the combined compositions can simultaneously act on different levels:

Degradation of late colonizer dominated dental biofilm (anaerobic shift to Aggregatibacter actinomycetemcomitans A.a., Porphyromonas gingivalis P.g., Treponema denticola T.d. content=Red Complex according to Prof. Socransky, J Clin Periodontol 1998; 25:134-44) by Chlorhexidine. Frustrate phagocytosis of a biofilm leads to overstimulation of inflammatory cytokines and shift of TH1 mediated immune defense (acute lesion) to TH2 dominated cytotoxic defense (chronic disease). The subgingival biofilm degradation and disturbance of regrowth can be achieved by the composition's chlorhexidine or chitosan component through disinfection and disturbance of the microbial cell wall synthesis.

Inhibition of microbial and immune system proteases (e.g. gingipains from P. g. and MMPs) which lead to disjunction and downregulation of cellular adhesive structures via E-Cadherin, opening paracellular spaces to infection and resulting in LPS induced inflammation in deeper connective tissue.

The compositions are designed to restore a non inflammatory state immune function and redifferentiate cellular adhesive structures to restore barrier function, the main component of innate immunity.

Immune modulation of the chronically shifted TH2 cytotoxic reaction due to cytokine overexpression (TNF-alpha, NO) as result of failed phagocytosis of biofilm protected anaerobic bacteria releasing LPS, blockade of MMP protease and osteoclast activation is essential to reduce the inflammatory response and tissue destruction in deeper connective tissues and bone. Sub- or low dose antimicrobiotical doses of immunomodulators like tetracycline are able to block MMP activation and macrophage/monocyte overstimulation. Bisphosphonate is able to block cholesterol synthesis and down regulate osteoclast stimulation and synthesis of inflammatory mediators. Cholesterol synthesis is regulated via mevalonate-dependent (MAD) route or isoprenoid pathway, which is an important cellular metabolic pathway that serves as the basis for the biosynthesis of molecules used in processes as diverse as terpenoid synthesis, protein prenylation, cell membrane maintenance, hormones, protein anchoring, and N-glycosylation. It is also a part of steroid biosynthesis.

To shift the TH2 driven immune response with resulting tissue destruction (periodontal ligament, alveolar bone, dermis, mucosa) back to a healthy, non inflammatory state it is necessary to redirect overstimulated inflammatory pathways, allow tissue clearance of inflammatory mediators and modulate immune response to stop chronic inflammation and destruction in deeper connective tissue and restore barrier function of the epithelial or endothelial structure.

Adhesive cellular complex rejunction and reestablishment of barrier function and closure of paracellular infective or immune system driven pathways into deeper tissues can be reached by inhibition of inflammatory and infective dedifferentiation (NFkB, MAPK, p38, p53, COX/PGE, caspase) and rejunction and differentiation in the tissues (ERK, PKC stimulation) by down regulation of cytokine overexpression via non-steroidal and antiinflammatory as well as immunomodulatory drug.

Surprisingly it has been shown according to the invention that the specific selection and combination of the components of the compositions results in a highly effective treatment regimen.

The inventive compositions containing chitosan and vitamin D3 or chlorhexidine and vitamin D3 as sole active compounds are able to stop inflammation. Alternatively, OPC can also be comprised as active compound.

According to a further alternative of the invention, ginger, curcumin and/or calcium or any derivatives thereof may also be comprised as active compounds in the compositions. The foregoing description will be more fully understood with reference to the following examples. Such examples are, however, merely representative of methods of practicing one or more embodiments of the present invention and should not be read as limiting the scope of invention.

### Examples

Examples to show efficiency and efficacy of the composition

Initial motivation and cleaning instruction, supragingival biofilm reduction by trimestrially repeated brushing in CHX solution 0.07 wt % for two weeks by the patient and tooth and pocket cleaning by ultrasonic scaling as well as covering exposed root dentine by adhesive fillings to hide protein coaggregational binding sites for anaerobic microbial species (A.a., P.g., T.d.) was administered.

Cases were treated with the composition comprising chlorhexidine (0.07 wt %), and vitamin D3 0.005 wt % for 10 days 2-5 times daily (case 1) or chlorhexidine (0.07 wt %), vitamin D3 0.025 wt % and OPC (10 wt %) for 3-13 days.

TABLE 2

| | Total Nr. of | | | | | |
|---|---|---|---|---|---|---|
| | sites measured | CPITN | | | | |
| | (6 per tooth)/ | Code | | Code 4: | | |
| | teeth | 3 + 4 Nr. | % | severe | % | Treatment time |
| Case 1 (J. B.) | Periodontitis | | | | | |
| Before treatment | 156/26 | 100 | 64 | 44 | 28 | |
| D3 0.0005 wt % + | 156/26 | 100 | 64 | 21 | 13.4 | 10 days |
| CHX 0.07 wt % | | | | | | code 4 -14.6% |
| formulation | | | | | | |
| CHX 0.07% wt % + | 156/26 | 75 | 48.10 | 0 | 0 | 1 week |
| D3 0.0025 wt % + | | | | | | code 3 + 4 -25%, |
| OPC 10 wt % | | | | | | code 4 -13.4% |
| case 2 (B. O.) | Periodontitis | 100 | | 75 | | |
| | 12/2 | | | | | |
| CHX 0.07% wt % + | | 58 | | 0 | | 14 days application |
| D3 0.0025 wt % + | | | | | | code 3 + 4 -42% |
| OPC 10 wt % | | | | | | code 4 -75% |
| case 3 (E. B.), | Periimplantitis | 83 | | 0 | | |
| | 6/1 | | | | | |
| CHX 0.07% wt % + | | 33 | | 0 | | 3 months application |
| D3 0.0025 wt % + | | | | | | code 3 + 4 -50% |
| OPC 10 wt % | | | | | | |
| case 4 (J. B. B) | Dentition | | | | | No inflammation |
| CHX 0.07 wt %- | difficilis | | | | | after 9 days of |
| D3 0.0025 wt %- | 18 | | | | | administration |
| OPC 10 wt % | | | | | | |
| cases 5, 6 | Gingivitis, | Inflammation | | | | No more edema after |
| (B. K, H. K.) | edema + | | | | | 9 resp. 13 days of |
| CHX 0.07 wt %- | Mc Calls | | | | | treatment |
| D3 0.0025 wt %- | girlande | | | | | |
| OPC 10 wt % | | | | | | |
| Case 7 (V. W.) | Healing cap | | | | | No mucosal |
| CHX 0.07 wt %- | placement | | | | | inflammation around |
| D3 0.0025 wt %- | | | | | | healing cap after |
| OPC 10 wt % | | | | | | 3 d application |
| Case 8 (W. G.) | Periodontitis | 79 | | 67.5 | | |
| | 120/20 | | | | | |
| | Miller Fischer | | | | | |
| | autoimmune | | | | | |
| | syndrome after | | | | | |
| | deep dive | | | | | |
| | accident | | | | | |
| CHX 0.07 wt %- | Periodontitis | 62.7 | | 51.9 | | 37 d 3 hopeless teeth |
| D3 0.0025 wt %- | 102/17 | | | | | had to be extracted |
| OPC 10 wt % | | | | | | |
| Case 9 (I. B.) | Inflamed | | | | | 3 weeks application |
| CHX 0.07 wt %- | extraction | | | | | no Inflammation, |
| D3 0.0025 wt %- | socket under | | | | | regeneration to |
| OPC 10 wt % | new bridgework | | | | | gingiva level of |
| | | | | | | socket, radio-graphic |
| | | | | | | bone gain in inflamed |
| | | | | | | area + 2-4 mm |
| Case 10 (A. G) | Gingivitis, | | | | | 4000 I.E. vit D3 |
| | Mc Calls | | | | | systemically 2 |
| | edema | | | | | months |
| | | | | | | Shallow pockets, |
| | | | | | | no edema, stop of |
| | | | | | | further attachment |
| | | | | | | loss |
| Case 11 (M. M.) | Periodontitis, | 61 | | 22 | | 4000 I.E. vit D3 |
| | Periimplantitis | | | | | systemically 2 |
| | 18/3 | | | | | months |
| | | 38 | | 22 | | some pocket |
| | | | | | | regeneration code 3 |
| | | | | | | (+33%), no edema, |
| | | | | | | no sensitivity, |
| | | | | | | stop of further |
| | | | | | | attachment loss |

The composition comprising CHX 1 wt % and vitamin D3 (0.0005 wt %) without and with OPC (cowberry standardized extract 10 wt %) is shown in cases 1 to 9.

The immunomodulatory capacity of the formulation containing CHX, and vitamin D3 (0.0005 wt %) was shown in case 1 where regeneration was observed only in class 4 CPITN pockets, reduction by 14.6% within 10 days of treatment (2-3 administrations/d) was observed but a complete disappearance of Mc Calls girlande representing the edema in gingivitis and disappearance of tooth sensibility could be demonstrated.

Deliberate regeneration took place in case 1 after application of a modified formulation containing CHX 0.07 wt %, vitamin D3 0.005 wt % and oligomeric proanthocyanids (OPC, cranberry extract) 10 wt % for only one week. 25 Class 3 pockets regenerated to class 1+2 (64-48.1%: +15.9%), all class 4 pockets where eliminated (reduction 13.4%)

A vitamin D3+OPC CHX gel formulation shown in cases 1-9 is therefore able to induce a comparable regeneration in the diseased periodontal pocket or inflamed extraction socket as well as around an implant healing cap. Systemic application of 40001.E. vit D3 over 2 months showed some regeneration in code 3 CPITN pockets, stop of edema, inflammation and attachment loss (cases 10, 11).

Results Using Chitosan and Vitamin D3 Compositions:

TABLE 3

| | | |
|---|---|---|
| Case 12 (M.K.) | Tooth | 6 sockets with full |
| Chitosan 1 wt % | extraction | epithelial coverage |
| Vit. D3 0.0025 wt % | 13-23 | after 3 days, bone |
| | | regeneration 30% |
| | | after 4 weeks |
| Case 12 control | Tooth | Epithelial coverage |
| | extraction | after 24 days, no |
| | 14, 15 | bone regeneration |
| | | after 4 weeks |
| Case 13 | Aseptic bone | No epithelial |
| | necrosis | coverage region 43 |
| | (complication | for 8 weeks |
| | after | without treatment |
| | implant loss | Epithelial coverage |
| | 43 and i.v. | after single |
| | Bisphosphonate | application |
| | treatment | Hydrogel Chitosan |
| | | 1 wt % Vit. D3 |
| | | 0.0025 wt % |

A chitosan+vitamin D3 gel formulation as shown in case 12 is able to induce deliberate regeneration of mucosal epithelium and maxillary bone after 4 weeks in comparison to untreated extraction sockets.

As a complication after implantation region 43 and antiosteoporotic i.v. Bisphosphonate treatment the implant 43 was lost leaving an area of infected aseptic bone necrosis class 2.

For eight weeks the area was not recovering and showed continuous suppuration and infection. After a single application of the chitosan 1 wt %/vitD3 0.0025 wt % hydrogel, full epithelial coverage and regeneration of the mucosa could be observed.

Hydrogel is a medical device comprising PEC (polyelectrolyte composite) wherein the polymers are connected via ionic binding. Chitosan is the cationic element thereof and carboxymethylcellulose (CMC) is the anionic part thereof. Chitosan is the only cationic biopolymer, CMC may be replaced in the hydrogel by alginate, pectin, heparin, hyaluronan, xanthane, carrageenan, sulfated cellulose, dextran sulfate and, for example, acetylated chitosans. Self-assemblance of the PEC skeleton is provided by 10-30% calcium phosphate.

1. A composition comprising chitosan, vitamin D and oligomeric proanthocyanid (OPC).

2. The composition of claim 1, wherein the vitamin D is in a form selected from the group consisting of cholecalciferol, calcifediol and calcitriol.

3. The composition of claim 1, wherein chitosan is present in an amount of from about 0.001% to 50% by weight of the composition, preferably from about 0.01% to 20% by weight of the composition, more preferably from about 0.1% to 10% by weight of the composition, and even more preferably about 1% by weight of the composition.

4. The composition of claim 1, further comprising chlorhexidine.

5. The composition of claim 4, wherein the chlorhexidine is present in an amount of from 0.01% to 10 wt %, preferably from 0.01% to 5 wt %, and more preferably from 0.1% to 2 wt %.

6. The composition of claim 1, wherein the vitamin D is present in an amount of from 0.00001% to 1% by weight of the composition, preferably from 0.00005% to 0.05% by weight of the composition, and more preferably from 0.0001% to 0.01% by weight of the composition.

7. The composition of claim 1, wherein the OPC is present in an amount of from 0.00001% to 50% by weight of the composition, preferably from 0.0001% to 20% by weight of the composition, more preferably from 0.001% to 10% by weight of the composition, and even more preferably about 10% by weight of the composition.

8. The composition of claim 1, further comprising ginger, curcumin and/or calcium.

9. The composition of claim 1, wherein the composition does not comprise a NSAID or bisphosphonate.

10. The composition of claim 1, wherein chitosan, vitamin D and oligomeric proanthocyanid are the sole active compounds.

11. The composition of claim 1, wherein said composition is in a form selected from the group consisting of a solid, a liquid and a gel.

12. The composition of claim 1, further comprising a pharmaceutically acceptable carrier, thereby forming a pharmaceutical composition.

13. (canceled)

14. A method of treating or preventing an oral infection or oral inflammation, comprising the step of administering the composition of claim 1 to a patient in need thereof.

15. A method of treating epithelial or mucosal wounds, comprising the step of administering the composition of claim 1 to a patient having an epithelial or mucosal wound.

16. The method of claim 14, wherein the composition is administered to a periodontal pocket.

17. The method of claim 14, wherein the oral infection or oral inflammation is associated with a condition selected from the group consisting of oral mucositis, gingivitis, periodontitis, and periimplantitis.

18. The method of claim 14, wherein the composition is administered in a dosage form selected from the group consisting of a gel, a hydrogel, a cream, and a solution.

19. The method of claim 15, wherein the composition is administered in a dosage form selected from the group consisting of a gel, a hydrogel, a cream, and a solution.
